(19) United States
(12) Patent Application Publication
(10) Pub. No.: US 2007/0179529 A1
(43) Pub. Date: Aug. 2, 2007

(54) Self-looping suture and method of using the same

(76) Inventor: Mark C. Doyle, San Diego, CA (US)

Correspondence Address: KNOBBE MARTENS OLSON & BEAR LLP, 2040 MAIN STREET, FOURTEENTH FLOOR, IRVINE, CA, 92614, US

(21) Appl. No.: 11/342,065

(22) Filed: Jan. 27, 2006

(51) Int. Cl. A61L 17/00

(52) U.S. Cl. 606/228

(57) ABSTRACT

Disclosed herein is a self-looping suture comprising a coiled section and a non-coiled section. Also disclosed herein is a method of suturing a tissue comprising obtaining a self-looping suture comprising a needle, a coiled section, and a non-coiled section, passing the needle and the coiled section of the self-looping suture through the tissue, grasping the non-coiled section of the self-looping suture with a grasper through the loop of the coiled section, and pulling the free end of the non-coiled section through the loop of the coiled section thereby forming a knot.

### BACKGROUND OF THE INVENTION

1. Field of the Invention

The present invention relates to an improved suture for use in surgery and methods of using the same.

2. Description of the Related Art

Endoscopic surgery is widely practiced. The advantages of reduced trauma and time have made this minimally invasive surgical technique popular. Because the surgery is performed inside the patient, via small incisions and through the use of long slender instruments, it does require relatively high levels of skill and training on the part of the surgeon. Suturing is particularly difficult, especially knot tying. Many surgeons cannot tie knots endoscopically, and many others find it unacceptably time consuming. Techniques of knot formation and tying have been developed, such as forming a loop of suture material by wrapping the suture thread around the tip of a grasping tool, but many surgeons still find it difficult if not impossible. This difficulty has led to the development of numerous devices intended to ease knot formation and tying.

One group of devices is composed of instruments intended to ease knot formation, including devices which attempt to duplicate the surgeon's natural hand motion in forming loops to tie a knot. Another group includes sutures which have a preformed partial knot through which the surgeon maneuvers the needle to complete the knot. The former devices are generally not found to be advantageous, while the latter group of devices are expensive. There exists a need for a low cost functional solution to the problem of forming and tying knots during endoscopic surgical procedures.

### SUMMARY OF THE INVENTION

Disclosed herein is a self-looping suture comprising a coiled section. Also disclosed herein is a method of suturing a tissue comprising obtaining a self-looping suture comprising a needle, a coiled section, and a non-coiled section, passing the needle and the coiled section of the self-looping suture through the tissue, grasping the non-coiled section of the self-looping suture with a grasper through the loop of the coiled section, pulling the free end of the non-coiled section through the loop of the coiled section and thereby forming a knot.

### BRIEF DESCRIPTION OF THE DRAWINGS

FIG. 1A shows an embodiment of a self-looping suture, where the coiled section is near the needle, and where the coiled section is wound clockwise. FIG. 1B shows an embodiment of a self-looping suture, where the coiled section is further away from the needle. FIG. 1C shows an embodiment of a self-looping suture having two loops in the coiled section. FIG. 1D shows an embodiment of a self-looping suture, where the coiled section is wound counterclockwise.

FIG. 2 shows an embodiment of a self-looping suture, where the sharpened end of the coiled section acts as a needle.

FIG. 3 shows an embodiment of a self-looping suture having a coiled section made of ductile material. FIG. 3A shows the suture being pulled through a tissue. FIG. 3B shows the loop of the coiled section of the suture becoming straight when tension is applied to the suture. The coiled section remains straight even when tension is removed, as shown in FIG. 3C. FIG. 3D shows that the loop in the coiled section is re-formed only after applying a slight reverse pressure.

FIG. 4A shows an embodiment of a self-looping suture with stacked loops. FIG. 4B shows an embodiment of a self-looping suture with spread loops. FIG. 4C shows an embodiment of a self-looping suture with spread loops separated by a non-coiled section.

FIG. 5 shows an embodiment of a self-looping suture having no non-coiled section.

FIGS. 6A-6F show the steps of an embodiment of a method of suturing an incision.

FIGS. 7A-7D show the steps of an alternative embodiment of a method of suturing an incision.

### DETAILED DESCRIPTION OF THE PREFERRED EMBODIMENTS

The term “suture” generally refers to a thread or fiber used in surgery to sew two tissue parts together. Sutures currently available in the art generally are connected to a needle by the manufacturer. Thus, the term “suture” also includes the combination of the fiber and the needle together.

In the first aspect, disclosed herein is a self-looping suture comprising a coiled section. In some embodiments, the suture further comprises a non-coiled section. In some embodiments, the suture further comprises a needle. By “self-looping” it is meant that the suture, in its laid-out state and without any tension applied on it, comprises at least one loop along the length of its thread.

In some embodiments, the coiled section is circular. In other embodiments, the coiled section is not circular. In some of these embodiments, the coiled section is oval, elliptical, substantially square, substantially triangular, or substantially polygonal.

In some embodiments, the non-coiled section is a thread or fiber used for suturing a tissue during or subsequent to surgery. The non-coiled section in general is the same as, or similar to, the thread or fiber part of a currently available suture.

In some embodiments, the coiled section is of a different material than the material that forms the non-coiled section. In some of these embodiments, the coiled section is made up of a material selected from the group consisting of, but not limited to, spring steel, stainless steel, nickel or nickel alloys, nitinol, beryllium copper, plastic, such as nylon, polyester, polypropylene and the like, fabric thread impregnated with a stiffening agent, such as glue, resin, and the like, and elastomeric materials, such as polyurethane, silicone, and the like, or a combination thereof.

In some embodiments, the coiled segment is part of a suture thread that has been modified. In some of these embodiments, a portion of the thread or fiber of the suture is coated with a coating to form the coiled section. Thus, the coated portion of the suture forms the coiled section and the non-coated portion of the suture forms the non-coiled section. In some of these embodiments, the coating is selected from the group consisting of, but not limited to, spring steel, stainless steel, nickel or nickel alloys, nitinol, beryllium copper, plastic, such as nylon, polyester, polypropylene and the like, fabric thread impregnated with a stiffening agent, such as glue, resin, and the like, and elastomeric materials, such as polyurethane, silicone, and the like, or a combination thereof.

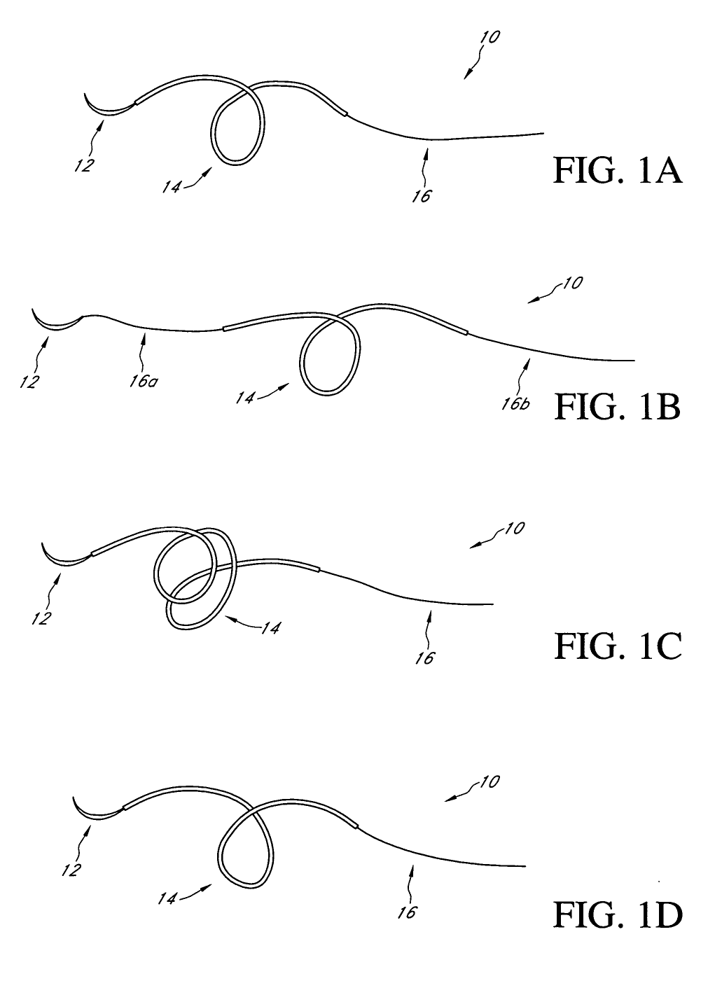

FIG. 1A shows a schematic of an embodiment of a suture disclosed herein. The suture 10 comprises three sections: a needle 12, a coiled section 14, and a non-coiled section 16. In some embodiments the cross-sectional thickness of the coiled section 14 is substantially the same as the cross-sectional thickness of the non-coiled section 16. In other embodiments, the coiled section 14 is thicker than the non-coiled section 16. In yet other embodiments, the non-coiled section 16 is thicker than the coiled section 14. The diameter of the loop in the coiled section 14 is preferably large enough to allow a grasper device to fit therethrough.

In some embodiments the loop of the coiled section is wound clockwise (FIG. 1A), whereas in other embodiments, the loop of the coiled section is wound counterclockwise (FIG. 1D).

While in the figures the needle 12 is shown as an arced needle, those of skill in the art recognize that the disclosed self-looping suture is configured to be used with a needle of any size or shape, whether arced, bent, or straight.

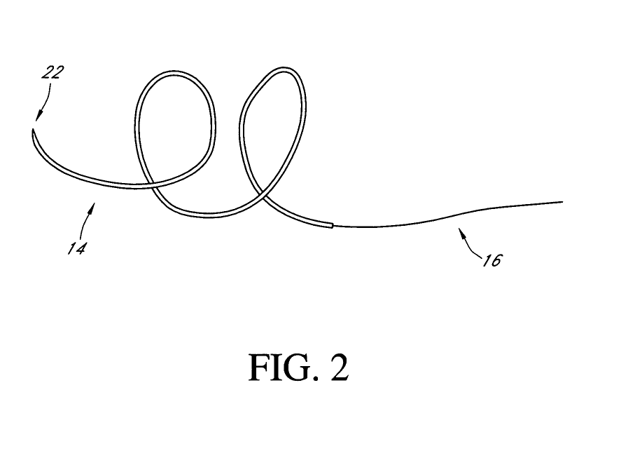

In some of these embodiments, the coiled section 14 is made up of a material that can be sharpened, such as metal or plastic. As shown in FIG. 2, the terminal part 22 of the coiled section 14 is sharpened to such extent that it can penetrate a tissue and act as a needle. These embodiments have the added advantage that they eliminate the need to have a separate needle attached to the suture.

In some embodiments, the coiled section 14 is near the needle 12. By near the needle it is meant that the needle is attached to the coiled section, or is within about an inch of the coiled section. FIG. 1A shows an example of a suture within these embodiments. In the embodiment shown, the coiled section 14 is directly connected to the needle 12. However, in some embodiments, as exemplified in FIG. 1B, a short, i.e., less than about one inch, piece of non-coiled thread 16a intervenes between the coiled section 14 and the needle 12.

In other embodiments, the coiled section 14 is further away from the needle 12. By “further away from the needle” it is meant that the needle is more than about an inch away from the coiled section. The example shown in FIG. 1B is within these embodiments when the length of the non-coiled section 16a is greater than one inch.

When the needle is passed through tissue, the surgeon pulls on the needle to pull a portion of the suture through the tissue as well. The pull on the needle creates a tension on the suture. In some embodiments, the coiled section 14 is flexible. In these embodiments, the tension on the suture causes the coiled section to become partially or substantially straight as it is going through the tissue. The straightening of the coiled section is to such a degree as to allow the suture, both the coiled and the non-coiled sections, to pass through the tissue with relative ease. In some of these embodiments, the coiled section is stiff enough such that the loop of the coiled section forms again once the tension is removed.

In some embodiments, the coiled section is stiff such that when the suture is pulled through the tissue, the coiled section does not straighten out.

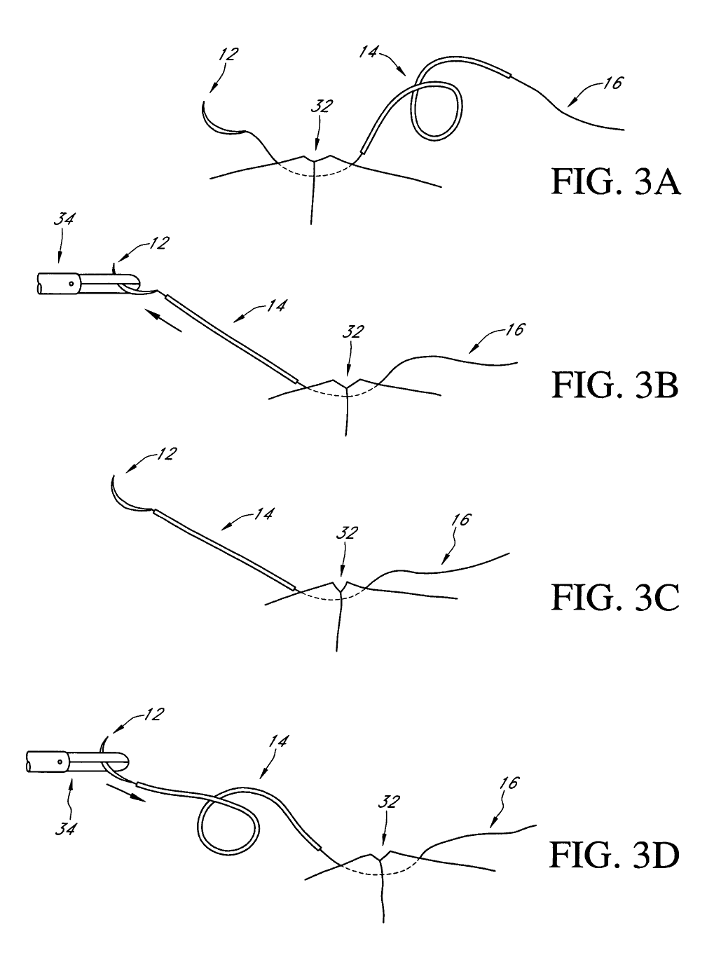

In some embodiments, the coiled section of the suture is ductile. FIG. 3 depicts one such embodiment. Prior to the application of tension to the suture, the coiled section exhibits at least one loop (FIG. 3A). Once tension is applied to the suture, for example by the use of a grasper 34, the coiled section becomes partially or substantially straight (FIG. 3B). However, once the tension is removed, the coiled section does not re-form itself. It rather remains partially or substantially straight (FIG. 3C). The surgeon can re-form the loop in the coiled section by applying a slight reverse pressure on the suture (FIG. 3D). In some embodiments, the surgeon can re-form the loop in the coiled section by changing the temperature of coiled section slightly.

Ductile material that can be used for the coiled section of the suture in these embodiments includes, but is not limited to, soft steel, aluminum, copper, monofilament polymer (such as polytetrafluoroethylene (PTFE), impregnated fiber, and the like), and memory metal (such as nickel-titanium alloy or Nitinol).

In some embodiments, the coiled section comprises a single loop. By a single loop it is meant that, going along the length of the thread, the thread makes only one 360 degree turn. FIG. 1A is an example of a suture that has a single loop. In other embodiments, the coiled section comprises two or more loops, i.e., two or more 360 degree turns along the length of the thread. FIG. 1C is an example of a suture that has two loops.

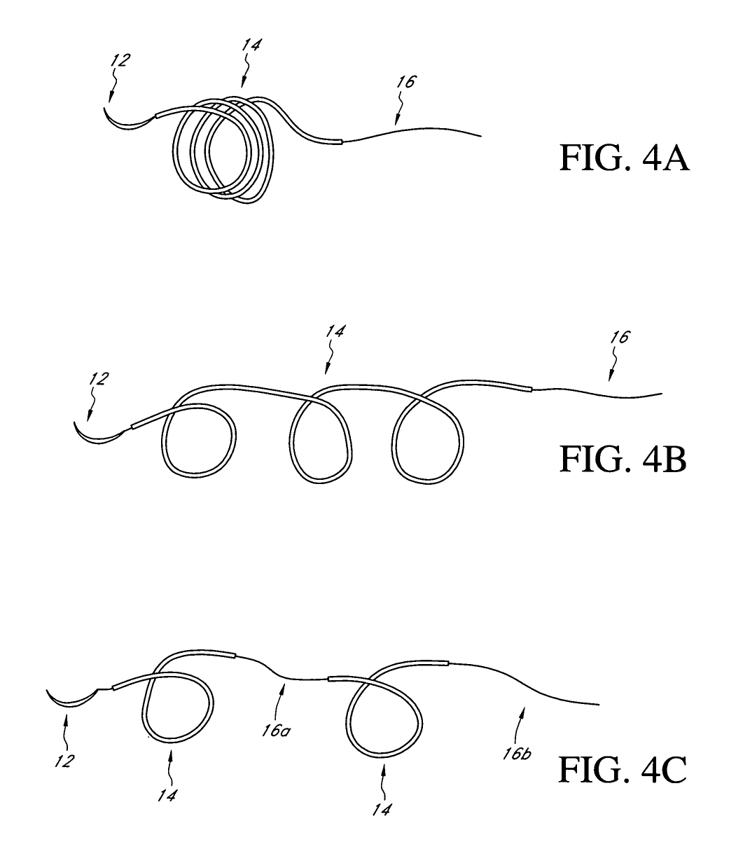

In some embodiments, the two or more loops are stacked, such as the loops found in a spring or a SLINKY® (FIG. 4A). In other embodiments, the two or more loops are spread along the length of the suture (FIG. 4B). In some embodiments, a non-coiled section 16a separates the various loops of the coiled section 14 (FIG. 4C).

In some embodiments, the coiled section of the suture comprises different sections having different properties. In these embodiments, the properties of the coiled section vary along the length of the suture. In some of these embodiments, the coiled section comprises two or more loops. One loop of the coiled section can be made up of one material and another loop can be made up of another material. In other embodiments, one loop of the coiled section is made up of thicker material than another loop. In yet other embodiments, the different sections of the coiled section are coated with different agents, or with different amounts of the same agent, to create the different properties. By varying the constitutive properties of the coiled section along the length of the suture, the tensile property of the coiled section is also varied. Thus, when the surgeon applies slight tension on the suture, one loop of the coiled section can straighten out, whereas another loop of the coiled section remains coiled. The surgeon can then apply greater tension for both, or all, loops to straighten out. In embodiments having greater than two loops in the coiled section, the surgeon can determine how many and which loops of the coiled section will be straightened out by varying the tension on the suture.

In some embodiments, the coiled section comprises the suture thread within a coiled sheath. In some embodiments, the sheath is a tubing. In some embodiments, the coiled sheath can be affixed to a first part of the thread, released from the first part of the thread, moved along the length of the thread to a second part of the thread, and affixed to the second part of the thread. In some embodiments, the coiled sheath can pass through a hole in the tissue created by the suture needle. Thus, in these embodiments, the coiled sheath is of such thickness that can follow the suture and the needle through the tissue. In other embodiments, the surgeon passes the needle and a non-coiled thread through the tissue and then passes the needle and a portion of the non-coiled section through a coiled sheath in order to create the coiled section from the portion of the non-coiled thread passed through the tissue. The surgeon can then place the coiled section in any desired location along the length of the suture. In some other embodiments, the surgeon can pass the end of the suture thread opposite the needle through a coiled sheath in order to create the coiled section from the portion of the non-coiled thread that has not passed through the tissue.

In some embodiments, the non-coiled thread is passed through the coiled sheath by the manufacturer or supplier and the sheathed thread is supplied as such to the surgeon. In some of these embodiments, the sheath is permanently affixed to a section of the thread, such as by heat shrinking, or other means. In other embodiments, the coiled sheath is supplied separately from the suture and the surgeon passes the thread through the sheath at the point of surgery, or while preparing for surgery.

In some embodiments, the coiled section of the suture is of a different color than the non-coiled section so as to enable the surgeon to quickly identify the coiled section.

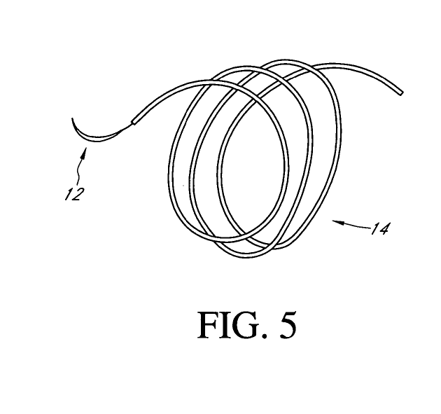

In another aspect, disclosed herein is a self-looping suture comprising a coiled section. In some embodiments, the suture further comprises a needle. In these embodiments, the self-looping suture does not comprise a non-coiled section. Instead, the entirety of the suture is comprised of a coiled section (FIG. 5). In some embodiments, the self-looping suture comprises one loop, whereas in other embodiments, such as the one shown in FIG. 5, the self-looping suture comprises more than one loop. The suture can comprise a separate needle, as shown in FIG. 5, or can have a sharpened portion that acts as a needle, as described above.

In conventional suturing techniques, the surgeon passes the needle of a suture through the tissue and pulls until there is a certain length of the suture thread on both sides of the incision. The surgeon then forms a loop with a grasper at the needle end of the suture, grasps the free end of the suture with the grasper and pulls the free end through the loop at the needle end, thereby forming a knot. It is very difficult for a surgeon to perform this procedure endoscopically, because it is difficult for the surgeon to form the loop at the needle end of the suture using the endoscopic instruments.

Thus, in another aspect, disclosed herein is a method of suturing a tissue comprising obtaining a self-looping suture comprising a needle, a coiled section, and a non-coiled section, passing the needle and the coiled section of the self-looping suture through the tissue, grasping the non-coiled section of the self-looping suture with a grasper through the loop of the coiled section, and pulling the free end of the non-coiled section through the loop of the coiled section thereby forming a knot, a half-knot, or a half-hitch. In some embodiments, the self-looping suture does not have a separate needle. In these embodiments, the free end of the coiled section is sharpened and acts as a needle.

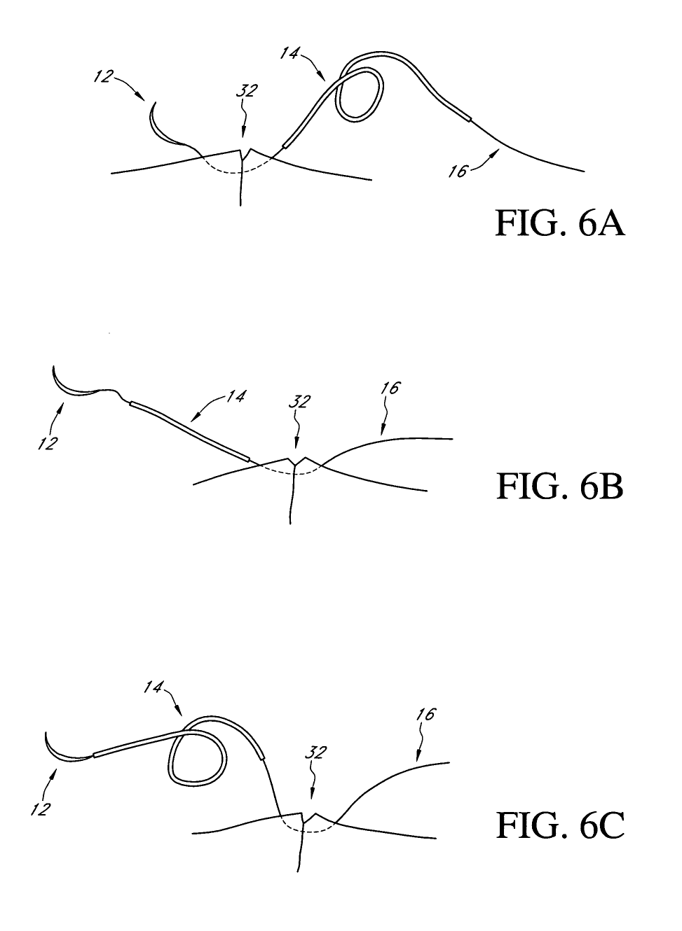

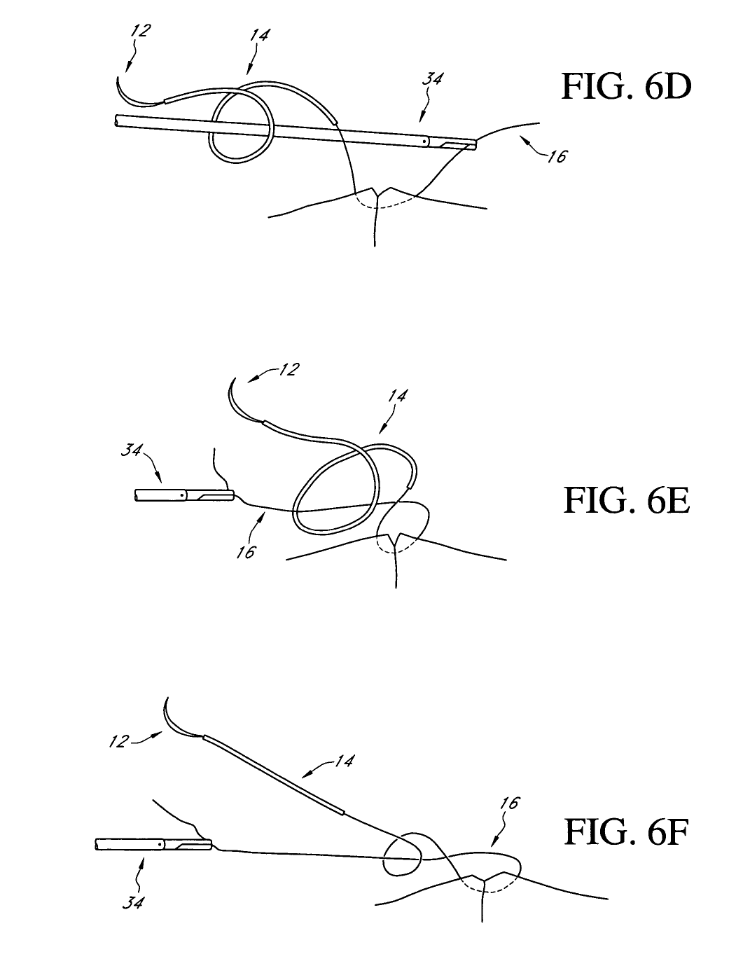

One embodiment of this method is depicted in FIGS. 6A-6F. The surgeon uses a grasper, such as a needle driver, to push the needle 12 through the target tissue 32 (FIG. 6A). The coiled section 14 of the suture is flexible enough so that as the surgeon pulls the thread through the tissue 32, it straightens out and acts like conventional thread as it passes through the tissue (FIG. 6B). Once the coiled section 14 has been pulled through the tissue 32, the surgeon relaxes the tension on the thread. This allows the coil to return to its natural loop shape (FIG. 6C). The surgeon now reaches through the loop of the coiled section 14 with a grasper 34 and grasps the free end 16 of the thread (FIG. 6D). The surgeon then pulls the free end 16 through the loop of the coiled section 14 (FIG. 6E). As the surgeon pulls on the thread, the loop of the coiled section is transferred downstream to the uncoiled section (FIG. 6F). When both ends of the suture are pulled tight, a basic knot, a half-knot, or a half-hitch is formed. The surgeon can easily determine the tension on the knot, and therefore the tightness with which the tissue is sutured.

In some embodiments, the self-looping suture does not have a separate needle. In these embodiments, one end of the coiled section is sharpened and acts as a needle.

In another aspect, disclosed herein is a method of suturing a tissue comprising obtaining a self-looping suture comprising a coiled section, passing the coiled section of the self-looping suture through the tissue, grasping the opposite end of the self-looping suture with a grasper through the loop of the coiled section, and pulling the opposite end of the suture through the loop of the coiled section thereby forming a knot, a half-knot, or a half-hitch. By “opposite end” it is meant the end of the suture that has not passed through the tissue.

In some embodiments, an end of the coiled section is sharpened and acts as a needle. In other embodiments, the self-looping suture has a separate needle. In some embodiments, the self-looping suture further comprises a non-coiled section.

In another aspect, disclosed herein is a method of suturing a tissue comprising obtaining a self-looping suture comprising a needle, a coiled section, and a non-coiled section, passing the needle through the tissue, grasping the needle with a grasper through the loop of the coiled section, and passing the needle through the loop of the coiled section thereby forming a knot, a half-knot, or a half-hitch. Thus, in this aspect, the coiled section is not pulled through the tissue. In some embodiments, the coiled section is further away from the needle; that is, a non-coiled section (FIG. 1B, 16a) intervenes between the needle and the coiled section.

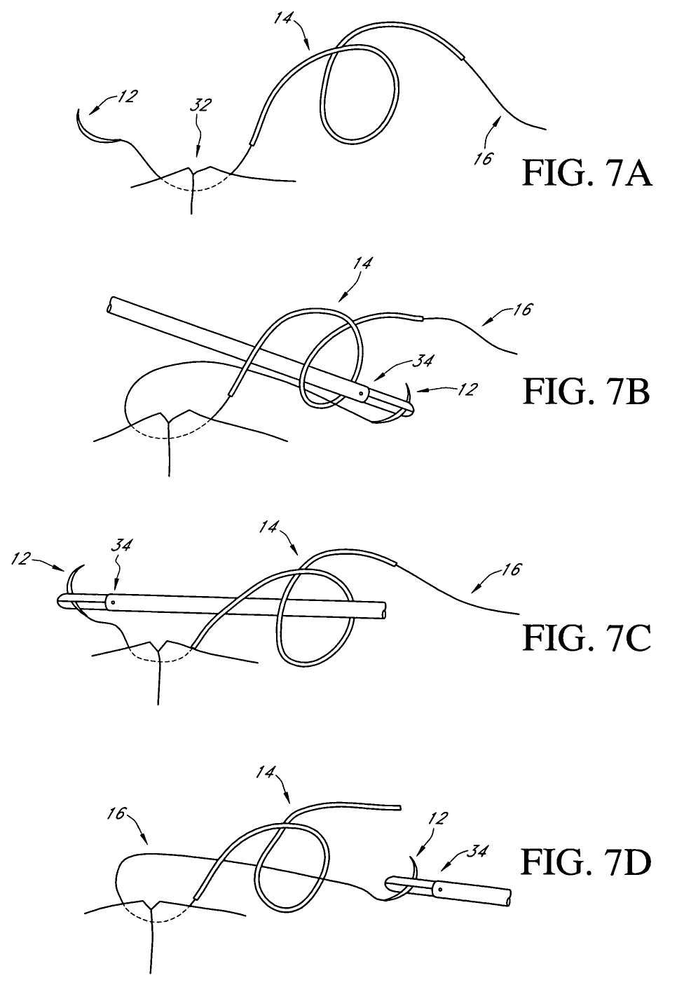

One embodiment of this method is depicted in FIGS. 7A-7D. The surgeon uses a grasper, such as a needle driver, to push the needle 12 through the target tissue 32 (FIG. 7A). The coiled section 14 of the suture remains on the opposite site of the tissue from the needle 12. At this point, the surgeon can pass the needle 12 through the loop of the coiled section 14 either with the needle driver, or with a grasper 34 (FIG. 7B). Alternatively, the surgeon can reach through the loop of the coiled section 14 with a grasper 34 and grasp the needle 12 (FIG. 7C). The surgeon then pulls the needle 12 through the loop of the coiled section 14 (FIG. 7D). As the surgeon pulls on the thread, the loop of the coiled section 14 is transferred upstream to the uncoiled section in between the needle 12 and the coiled section 14. When both ends of the suture are pulled tight, a basic knot, a half-knot, or a half-hitch is formed. The surgeon can easily determine the tension on the knot, and therefore the tightness with which the tissue is sutured.

In another aspect, disclosed herein is a method of suturing a tissue comprising obtaining a self-looping suture comprising a coiled section having a sharpened end, passing the sharpened end of the coiled section through the tissue, passing the sharpened end of the coiled section through the loop of the coiled section, and pulling the sharpened end through the loop of the coiled section thereby forming a knot, a half-knot, or a half-hitch. Thus, in this aspect, the coiled section is not pulled through the tissue.

It will be understood by those of skill in the art that numerous and various modifications can be made without departing from the spirit of the present invention. Therefore, it should be clearly understood that the forms of the present invention are illustrative only and are not intended to limit the scope of the present invention.

What is claimed is:

1. A self-looping suture comprising a coiled section.

2. The self-looping suture of claim 1 further comprising a non-coiled section.

3. The self-looping suture of claim 1 further comprising a needle.

4. The self-looping suture of claim 1, wherein said coiled section is of a different material than the material that forms said non-coiled section.

5. The self-looping suture of claim 1, wherein said coiled section is of a material selected from the group consisting of spring steel, stainless steel, nickel, nickel alloys, nitinol, beryllium copper, plastic, fabric thread impregnated with a stiffening agent, elastomeric material, and a combination thereof.

6. The self-looping suture of claim 5, wherein said plastic is selected from the group consisting of nylon, polyester, polypropylene,

7. The self-looping suture of claim 5, wherein said stiffening agent is selected from the group consisting of glue, and resin.

8. The self-looping suture of claim 5, wherein said elastomeric material is selected from the group consisting of polyurethane and silicone.

9. A self-looping suture comprising a thread, wherein a portion of said thread is modified to form a coiled section, and a portion of said thread is unmodified to form a non-coiled section.

10. The self-looping suture of claim 9, wherein a portion of said thread is coated with a coating to form said coiled section.

11. The self-looping suture of claim 10, wherein said coating is selected from the group consisting of spring steel, stainless steel, nickel, nickel alloys, nitinol, beryllium copper, plastic, fabric thread impregnated with a stiffening agent, elastomeric material, and a combination thereof.

12. The self-looping suture of claim 1, wherein said coiled section is flexible, such that tension on said suture causes a loop in said coiled section to become partially or substantially straight.

13. The self-looping suture of claim 12, wherein said loop in said coiled section forms again once said tension is removed.

14. The self-looping suture of claim 1, wherein said coiled section comprises a single loop.

15. The self-looping suture of claim 1, wherein said coiled section comprises two or more loops.

16. The self-looping suture of claim 3, wherein said needle is attached to said coiled section.

17. The self-looping suture of claim 3, wherein said needle is within about an inch of said coiled section.

18. The self-looping suture of claim 3, wherein said needle is more than about an inch away from said coiled section.

19. A method of suturing a tissue comprising
obtaining a self-looping suture comprising a coiled section and a non-coiled section,
passing said coiled section of said self-looping suture through said tissue,
passing said non-coiled section of said self-looping suture through a loop of said coiled section, and
pulling a free end of said non-coiled section through said loop of said coiled section thereby forming a knot.

20. The method of claim 19, wherein said self-looping suture further comprises a needle.

21. A method of suturing a tissue comprising
obtaining a self-looping suture comprising a coiled section having a sharpened end,
passing said sharpened end of said coiled section through said tissue, and
passing said sharpened end of said coiled section through at least one loop of said coiled section, thereby forming a knot.

22. A method of suturing a tissue comprising
obtaining a self-looping suture comprising a coiled section having a sharpened end,
passing said sharpened end of said coiled section through said tissue,
passing said sharpened end of said coiled section through at least one loop of said coiled section, and
pulling said sharpened end of said coiled section through said at least one loop of said coiled section thereby forming a knot.
